(19) United States
(12) Patent Application Publication
(10) Pub. No.: US 2003/0144683 A1
(43) Pub. Date: Jul. 31, 2003

(54) Inflatable members having concentrated force regions

(75) Inventors: Motasim Sirhan, Sunnyvale, CA (US); Debashis Dutta, Santa Clara, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Avantec Vascular Corporation, Sunnyvale, CA

(21) Appl. No.: 10/317,846

(22) Filed: Dec. 11, 2002

Related U.S. Application Data
(60) Provisional application No. 60/340,461, filed on Dec. 13, 2001.
(60) Provisional application No. 60/343,118, filed on Dec. 21, 2001.

(51) Int. Cl. A61M 29/00

(52) U.S. Cl. 606/194

(57) ABSTRACT

A device and a method using the same, for the treatment of stenosed areas during or prior to dilation, comprising at least one region along an expandable member which is configured to deliver a concentrated force to the diseased tissue site.

### CROSS-REFERENCES TO RELATED APPLICATIONS

This application claims the benefit of Provisional Application No.60/340,461 (Attorney Docket No. 020460-001700US), filed Dec. 13, 2001, and of Provisional Application No. 60/343,118, (Attorney Docket No. 020460-001710US), filed Dec. 21, 2001, both assigned to the same assignee as the present invention, and both incorporated herein by reference in their entirety.

### BACKGROUND OF THE INVENTION

1. Field of the Invention

The present invention relates generally to medical devices and methods, and more particularly to devices and methods for dilation or recanalization of a diseased vessel.

In percutaneous transluminal coronary angioplasty (PTCA) procedures, a guiding catheter is advanced until the distal tip of the guiding catheter is seated in the ostium of a desired coronary artery. A guidewire, positioned within an inner lumen of a dilatation catheter, is first advanced out of the distal end of the guiding catheter into the patient's coronary artery until the distal end of the guidewire crosses a lesion to be dilated. Then the dilatation catheter having an inflatable balloon on the distal portion thereof is advanced into the patient's coronary anatomy, over the previously introduced guidewire, until the balloon of the dilatation catheter is properly positioned across the lesion.

Once properly positioned, the dilatation balloon is inflated with inflation fluid one or more times to a predetermined size at relatively high pressures (e.g. 4-12 atmospheres) so that the stenosis is compressed against the arterial wall and the wall expanded to open up the passageway. Generally, the inflated diameter of the balloon is approximately the same diameter as the native diameter of the body lumen being dilated so as to complete the dilatation but not to significantly overexpand the arterial wall. Expansion of the balloon against the vessel wall can cause trauma to the vessel wall. After the balloon is finally deflated, blood flow resumes through the dilated artery and the dilatation catheter can be removed therefrom.

In such angioplasty procedures, there may be restenosis of the artery, i.e. reformation of the arterial blockage, which necessitates either another angioplasty procedure, or some other method of repairing or strengthening the dilated area. Often, the restenosis may be initiated by the injury caused to the vessel during the dilation process, which in part is due to the pressures (2-20 atm) applied to overcome the elastic recoil of the tissue at the stenosed area.

To overcome this problem, physicians sometime use blades mounted on the balloons to cut or incise the stenosis. However, there are several drawbacks to these devices. The blades themselves may cause injury to the artery as the catheter is moved through the artery. The blades are mounted on top of the balloon surface and are exposed to the artery. The blades are typically metal and attached to the balloon by welding, adhesives, fasteners, etc. While the attachments are made quite secure, there is always a risk that a particular attachment may fail, exposing the patient to significant risk if the blade detaches.

Accordingly, it would be a significant advance to provide improved devices and methods for treating a stenosed area to reduce the occurrence of restenosis or to provide effective dilation of the stenosis. In particular, it would be advantageous to provide improved methods and designs for securing blades and similar structures to angioplasty and similar balloon devices. This invention satisfies at least some of these and other needs.

### BRIEF SUMMARY OF THE INVENTION

The present invention is directed to intracorporeal devices and methods using the same, such as interluminal devices including catheters. The devices of the present invention may be used as balloon catheters for the treatment of stenosed areas during or prior to dilation.

In an embodiment, the devices of the present invention comprise at least one region along an expandable member, such as an expandable balloon, which is configured to deliver a concentrated force to the diseased tissue site. The concentrated region may be along either or both the longitudinal and transverse axis of the balloon. The concentrated region may be configured to apply continuous or intermittent concentrated force along the balloon. In an embodiment, the regions deliver a higher pressure to the select tissue sites due to relatively smaller contact surface between the expandable member and the tissue site.

In an embodiment, the balloon includes at least one, usually a plurality of wings along at a least a portion of its length, usually its working length, extending radially outward upon expansion of the balloon. The one or plurality of wings may extend along the entire circumference of the balloon, or only a portion of thereof.

In one embodiment, at least a portion of the wing is relatively stiffer than the rest of the balloon. In an exemplary embodiment, the relatively higher stiffness may be achieved by heat treatment of the wing for a period of time. By way of example, the heat treatment, may crystallize the balloon material along the wings. Upon expansion of the balloon, the wing areas will exert a concentrated force to the tissue, thus pushing back or excising the stenosis, or relieving stress in the stenosed area so that it is easier to dilate the vessel.

In another embodiment, the wing portion is melted, creating a solid crease or fold upon cooling of the material.

In an alternate embodiment, an adhesive, such as a UV curable adhesive, is introduced into the wings area (e.g., by way of injection) from the inner surface of the balloon. The fold region of the balloon including the adhesive is then exposed to UV energy, thus solidifying the adhesive in the fold and creating a stiffer concentrated region. Other suitable adhesives may also be utilized, such as epoxies, urethanes (e.g., non-UV curable), and cyanoacrylates.

In one embodiment, objects, usually relatively thin objects such as blades or wires, are introduced to the inside of the balloon to create the regions configured to deliver concentrated force. In an embodiment, the objects may be disposed within an existing crease or one to be created subsequent to the insertion of the object within the balloon. The objects may be formed from any suitable material, including but not limited to: adhesives, metals including stainless steel, metal alloys, liquid crystal polymers (LCPs), or composites. The objects may have any suitable shape including circular, rectangular, oblong, serpentine, helical, or combinations thereof.

In yet other embodiments, the object may be inserted and disposed longitudinally or radially along the interior of the balloon. Upon the expansion of the balloon, the regions including the object will exert a concentrated force to the luminal tissue in which the balloon is disposed. The object may comprise one or more objects. The one or more objects may have a continuous or intermittent dimension. The objects may be used alone or in combination with the foregoing adhesives.

The expandable members constructed according to the present invention will usually have a relatively integral or congruent exterior surface. By “integral,” it is meant that the wing formed in or on the balloon are formed of the same material as the balloon itself or, in some cases, from a comparable material which may be welded, glued, or melted into the balloon to form a continuous junction or interface that will not separate under any foreseeable conditions of use. Usually, the wing will be formed by molding, where the wing is formed as an element or part of the molded balloon structure. When formed by molding, the wing will usually be formed from the same material as the balloon, although the density, molecular weight, or other physical characteristics of the wing may vary relative to the material of the balloon. Alternatively, the wing may be formed separately from the same or a comparable polymer, where the wing is then attached by gluing, heat welding, ultrasonic welding, melting, or the like. Optionally, the wing may be heat-treated or otherwise reconfigured to change it's hardness, density, or other physical property as described elsewhere herein. The exterior surface will usually be free of abrupt changes and/or discontinuities in its exterior profile. Usually, the most exterior surface of the expandable member will be formed from the same material or may be formed from different materials. In an embodiment when the exterior surface material is formed from different material, the material will have, or is processed to have, similar properties.

### BRIEF DESCRIPTION OF THE DRAWINGS

FIG. 1 is an elevational, partially enlarged, side view of a balloon catheter embodying features of the invention.

FIG. 2A is an elevational, side view of an embodiment of a balloon embodying features of the invention.

FIG. 2B is a transverse cross-sectional view of the balloon of FIG. 2A taken along line 2B-2B.

FIGS. 2C and 2C are transverse cross sectional views of alternate embodiments of the balloon of FIG. 2B.

FIGS. 2E and 2F are elevational side views of alternate embodiments of a balloon embodying features of the invention.

FIGS. 3 and 4 are side and cross sectional, partially in section, views of an exemplary mold for making the balloons of the present invention.

FIGS. 5 through 10 are side and cross sectional, partially in section, views of an alternate embodiment of a balloon according to the present invention.

### DETAILED DESCRIPTION OF THE INVENTION

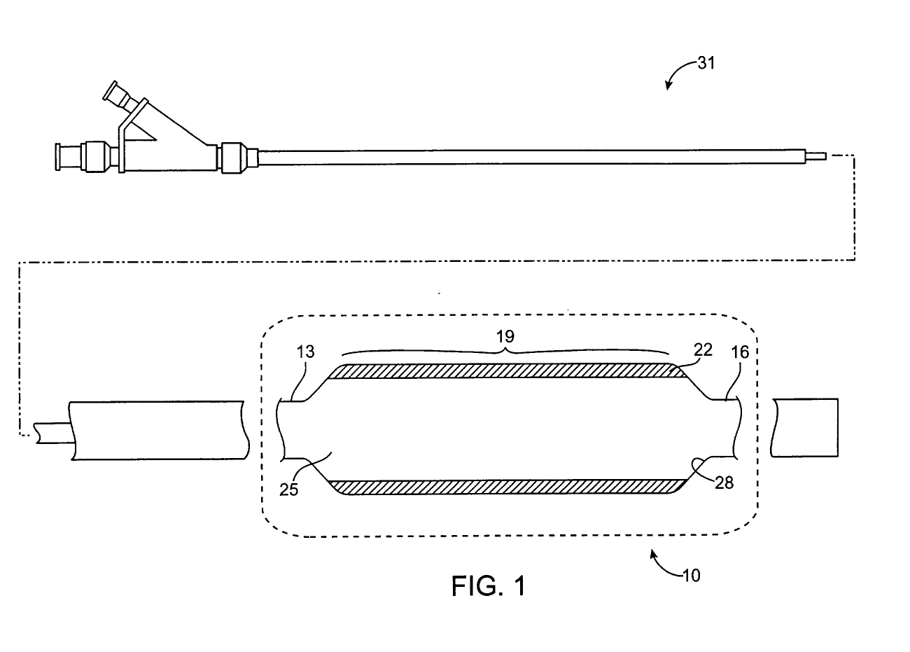

FIG. 1 illustrates a balloon 10 embodying features of the present invention, and generally having proximal and distal ends 13 and 16, a working length 19 extending along at least a portion therebetween, outer surface 22, and an inner chamber 25 defined by a balloon inner surface 28. The balloon 10 is usually used as part of a balloon catheter 31.

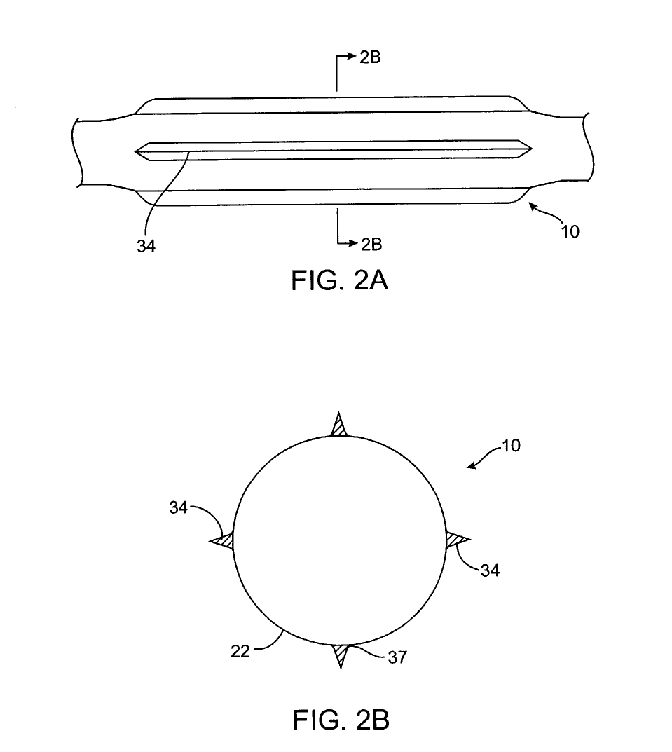

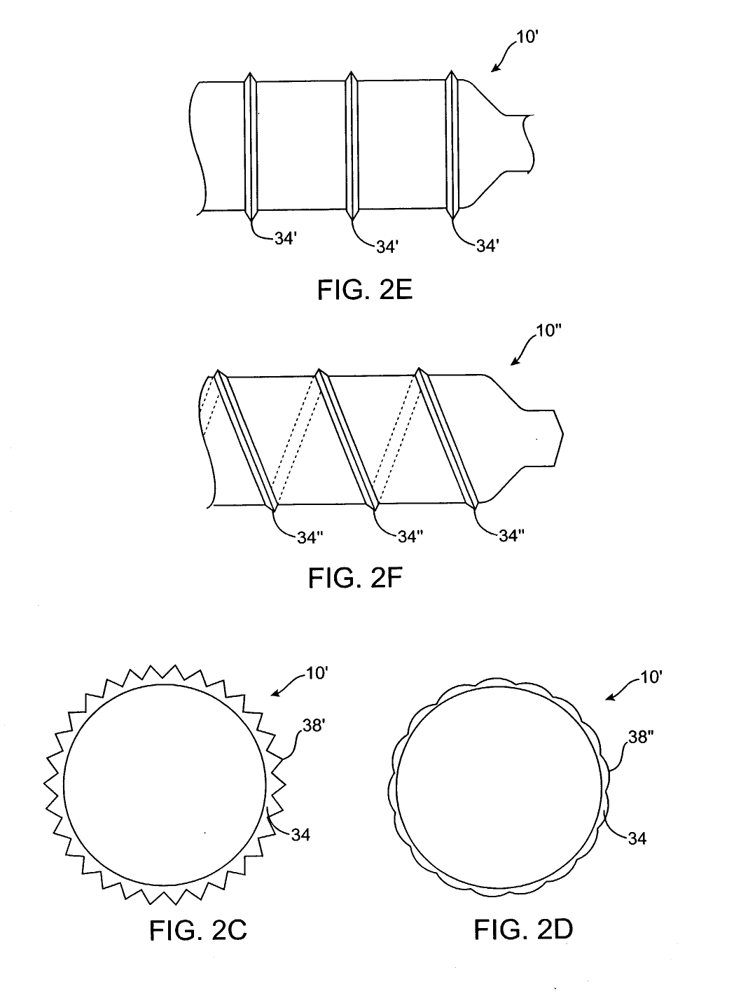

Now referring to FIGS. 2A through 2B, balloon 10 includes at least one wing 34, usually a plurality of wings 34, disposed along at least a portion of the length of the balloon, normally along the working length and usually being radially set apart, often at radially equidistance intervals. The wings 34 may be present in any number, and may be of any size, or form any suitable angle with a base 37 formed with reference to the outer surface of the balloon in an expanded configuration, as may be necessary to properly perform an intended procedure.

In some embodiments, the wings may have sharp or smooth apecies, as shown in FIGS. 2C and 2C as 38′ and 38″, respectively, or any combination thereof.

In another embodiment features of which are shown in FIG. 2E, the wings 34′ extend radially around the balloon 10′ and are longitudinally set apart. Each wing 34′ has a short dimension along the longitudinal axis of the balloon (for example more like a collar), with a plurality of such wings 34′ extending along at least a portion of the length of the balloon. The wings 34′ may be discrete wings, as shown in FIG. 2E, or may be in the form of a helical wing 34″ as shown in FIG. 2F.

The wings have a stiffness relatively higher than that of the balloon material adjacent thereto. The relatively higher stiffness wing provides regions providing concentrated force to the tissue upon expansion of the balloon within a diseased or stenosed vessel.

The relatively higher stiffness wings may be formed by way of any one or more of the methods and configuration described further below.

In the embodiment features of which are shown in FIGS. 1 and 2 portions of the balloon for forming the wing areas are exposed to a source of energy, such as a heat source, and are usually, crystallized or melted, and upon cooling (as the case may be) exhibit higher stiffness. The wings of the balloon may be then folded in the conventional manner to reduce the profile of the balloon prior to the introduction of the balloon to the lumen. Upon expansion of the balloon the wings open providing regions along the balloon able to provide a concentrated force to the stenosed area. The stenosed regions are then incised and/or pushed back against the wall without necessarily, but usually, bringing the total working length of the balloon into contact with the luminal wall and/or apply pressure thereto.

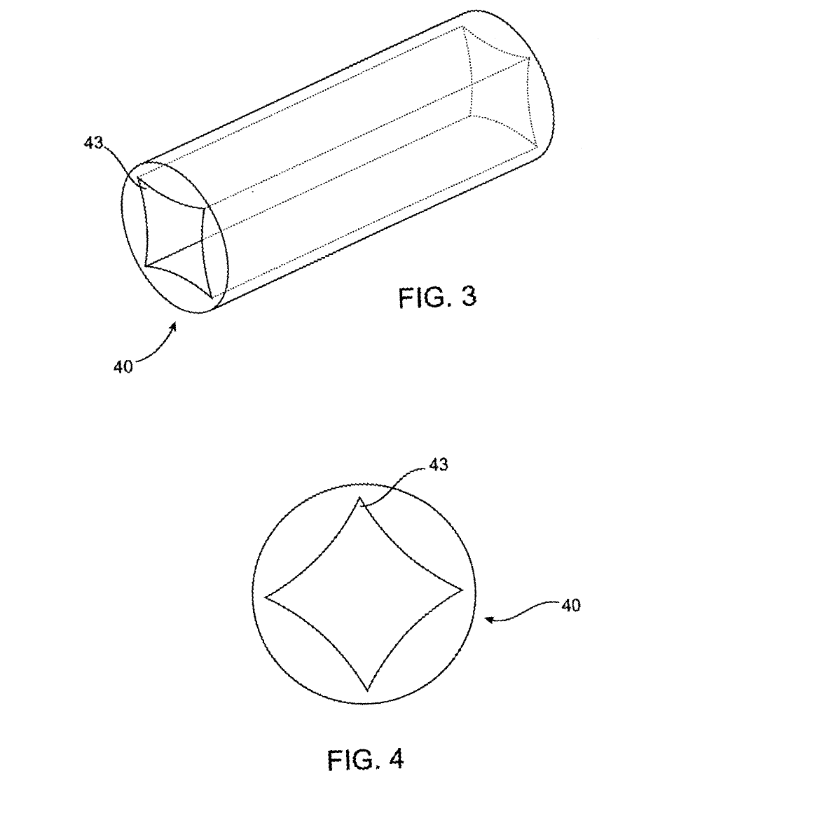

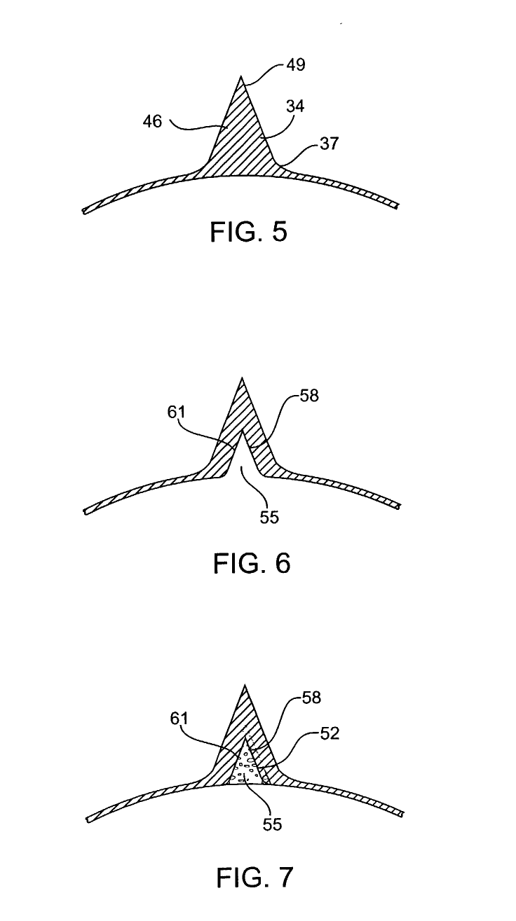

By way of example, relatively stiff polymeric material having sharp edges can act as a cutting surfaces. Balloons such as those formed from PET, nylon, polyimides, polyamides, polyurethanes with high durometer, blends or copolymers thereof, polymer blends (e.g., blends with fibers, composites, or other polymer(s)) may be formed to have creases using molds, such as mold 40 features of which are shown in FIGS. 3 and 4, with portions 43 corresponding to the balloon wings. The extreme ends of the creases 46 and 49 formed in the balloon, as shown in FIG. 5, are melt-pressed together in a clamp to create a sharp edge, having an exemplary height of about 0.002 inch to about 0.020 inch, and an exemplary width of about 0.002 inch to about 0.030 inches. Optionally, the top of the edge may further be sharpened with profile reducing devices such as sanding machine or laser.

In another embodiment features of which are shown in FIG. 7, an adhesive material 52 is disposed between a pocket 55, FIG. 6, formed between the inner sides, 58 and 61, of the wings creating a solid wing portion upon at least partial solidification of the wing. The wing may include a pocket formed between the inner sides of the wing which includes a material formed from another material such as an adhesive material. The adhesive is preferably a flexible or soft adhesive, such as polyurethane or UV-curable acrylates. The adhesive, by way of example, may be disposed in the creases or the grooves of the balloon interior surface before sealing the edges in a hot clamp, or may optionally be processed subsequently. The adhesive forms a wide base for the sharp extreme edge of the blade. The base provides support to the blade as it presses against the arterial wall. The suitable adhesives further included UV-curable acrylates, epoxies, polyurethanes (including non-UV curable), and cyanoacrylates.

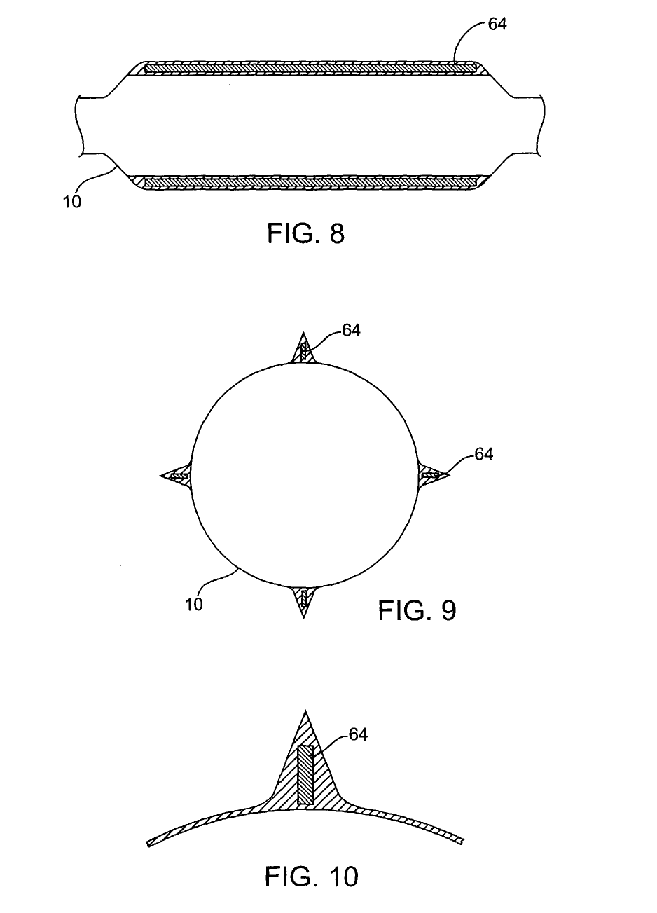

In another embodiment features of which are shown in FIGS. 8 through 10, an object such as a cutting element 64 may be disposed within the crease prior to sealing of the inner sides of the crease. The cutting element may be a blade having or a wire or any other suitable element. The cutting elements may be used with or without the use of the adhesive material in the pocket 55.

In yet other embodiments, the object may be inserted longitudinally or radially along the interior of the balloon. Upon the expansion of the balloon, the regions including the object will exert a concentrated force to the luminal tissue in which the balloon is disposed.

### EXAMPLES

### Example 1

Balloons formed from PET or Nylon12 (3.0×20 mm) were blown in the rectangular star shaped mold 40. Blades were made by flattening a 0.003 inch diameter stainless steel wire to a blade having width and depth dimensions of 0.002 inches and 0.005 inches, respectively. The length of the blade was sized to be about the same as the balloon working length. The balloons were creased along the edge of the rectangle in the axial direction to form inverse grooves in the interior surface of the balloon. The flattened wires or blades were introduced from the proximal end to the balloon interior chamber. One blade was placed within the creased groove and the edges of the grooves melt pressed with a heated clamp. The temperature of the clamp was adjusted so as to soften and/or melt the balloon material. Sufficient pressure was applied on that edge to press the two folds together, embedding the blade inside it. In one embodiment, a UV curable adhesive was placed on the groove to improve the retention of the blade in the groove. Three to four blades were placed in each balloon. In another embodiment, the edges were sanded down with sand paper to sharpen the edges. In some of the samples, the wires or blades at least partially protruded through the balloon material, while some were totally covered by the balloon material. Upon the inflation of the balloon, the edges with embedded blades had a more outwardly profile and could function as atheretomes. The blades, in the example, were not directly exposable to the arterial wall, thus reducing the likelihood of injury to the vessel. The balloons constructed according to the present invention demonstrated a better trackability as compared to standard cutting balloon having blades externally mounted to the balloon.

### Example 2

Balloons formed from PET and Nylon were blown using the rectangular mold 40 of FIG. 40, forming four inverted grooves in the balloon surface. The extreme edges of the grooves (e.g., 0.005 inches from the top) of the grooves were melt pressed in a clamp to create sharp edges. The height of the edges were approximately 0.005 inches with a width of about 0.002 inches. The top of the edges were further sharpened with a sand paper to form the surfaces to exert concentrated force. Upon inflation of the balloon, the melted edges had a more radially outward profile to act as cutting edges. Since the edges were part of the balloon material itself, the balloon was very flexible and trackable, compared to conventional balloons incorporating stainless steel blades on the outer surface.

Although certain preferred embodiments and methods have been disclosed herein, it will be apparent from the foregoing disclosure to those skilled in the art that variations and modifications of such embodiments and methods may be made without departing from the true spirit and scope of the invention. Therefore, the above description should not be taken as limiting the scope of the invention which is defined by the appended claims.

What is claimed is:

1. A device for dilating or recanalizing a vessel, comprising:
a balloon catheter comprising an expandable member having an outer surface and an inner chamber defined by an inner surface; and
at least one region along the expandable member integrally formed within the balloon and configured to deliver concentrated force to an interior of the vessel upon expansion of the expandable member in the vessel

2. A device as in claim 1 wherein the expandable member is a balloon.

3. A device as in claim 2 wherein the region extends along a longitudinal axis of the balloon.

4. A device as in claim 2 wherein the region extends along a radial axis of the balloon.

5. A device as in claim 2 wherein the region extends along a longitudinal axis and a radial axis of the balloon.

6. A device as in claim 1 wherein the region extends along a working length of the balloon disposed longitudinally between a balloon proximal end and a balloon distal end.

7. A device as in claim 1 wherein the region has a continuous dimension.

8. A device as in claim 1 wherein the region has an intermittent dimension.

9. A device as in claim 2 wherein the balloon includes at least one wing.

10. A device as in claim 9, wherein the wing is molded from the same material as the balloon.

11. A device as in claim 9 wherein the balloon includes a plurality of wings.

12. A device as in claim 9 wherein the at least one wing has a contour extending more radially outward upon the expansion of the balloon relative to the balloon.

13. A device as in claim 9 wherein the at least one wing includes portions having a relatively higher stiffness than the rest of the balloon.

14. A device as in claim 9 wherein the wing has a pocket.

15. A device as in claim 14 wherein the pocket is sealed from the interior chamber of the balloon.

16. A device as in claim 14 wherein the wing pocket includes a concentrating element disposed therein.

17. A device as in claim 16 wherein the concentrating element is a blade.

18. A device as in claim 16 wherein the concentrating element device is a wire.

19. A device as in claim 16 wherein the concentrating element is a tube.

20. A device as in claim 16 wherein the concentrating element has a continuous dimension.

21. A device as in claim 16 wherein the concentrating element has an intermittent dimension.

22. A device as in claim 17 wherein the concentrating element has a continuous dimension.

23. A device as in claim 17 wherein the concentrating element has an intermittent dimension.

24. A device as in claim 1 wherein the region includes a concentrating element.

25. A device as in claim 24 wherein the concentrating element is disposed within the interior chamber of the expandable member.

26. A device as in claim 25 wherein the concentrating element is fixedly attached to the inner surface of the expandable member.

27. A device as in claim 25 wherein the concentrating element is removably disposed within the inner chamber.

28. A device as in claim 25 wherein the concentrating element is removably attached to the inner surface.

29. A device as in claim 24 wherein a concentrating element is disposed within the wing.

30. A device as in claim 24 wherein the concentrating element is fixedly attached to an inner surface of the wing.

31. A device as in claim 24 wherein the concentrating element is removably disposed between an opposing inner surfaces of the wing.

32. A device as in claim 24 wherein the concentrating element is removably attached to an inner surface of the wing.

33. A device as in claim 24 wherein the concentrating element is configured to expand with the expandable member.

34. A device as in claim 25, wherein the region exterior surface is formed from the same material as the expandable member.

35. A device as in claim 24 wherein the concentrating element at least partially protrudes from the balloon material.
